(19) United States
(12) Patent Application Publication
(10) Pub. No.: US 2023/0356455 A1
(43) Pub. Date: Nov. 9, 2023

(54) Method for Producing a Mold Element for Producing Micro-Arrays, and Mold Element

(71) Applicant: LTS Lohmann Therapie-Systeme AG, Andernach (DE)

(72) Inventors: Michael Kulik, Urmitz (DE); Olga Grünwald, Koblenz (DE); Philip Roos, Kettig (DE); Nikolaj Tissin, Sinzig (DE)

(21) Appl. No.: 18/246,927

(22) PCT Filed: Aug. 31, 2021
(86) PCT No.: PCT/EP2021/074024

§ 371 Date: Mar. 28, 2023

(30) Foreign Application Priority Data
Sep. 30, 2020 (DE) 10 2020 125 484.9

(51) Int. Cl. B29C 59/04; B29C 59/02; B29C 33/38

(52) U.S. Cl. CPC B29C 59/04; B29C 59/022; B29C 33/3842; B29C 2059/023; B29L 2031/7544

(57) ABSTRACT

The invention relates to a method for producing a mold element for producing micro-arrays. First, a planar base element, in particular in the form of a film, is provided. Recesses are made in, in particular embossed into, an upper face of the element. The recesses are opened on a lower face of the base element in particular by using laser radiation. The invention also relates in particular to a mold element produced by means of this method, which mold element has recesses like the base element. Each of the recesses has openings in the lower face of the base element.

### CROSS-REFERENCE TO RELATED APPLICATIONS

This application is the United States national phase of International Application No. PCT/EP2021/074024 filed Aug. 31, 2021, and claims priority to German Patent Application No. 10 2020 125 484.9 filed Sep. 30, 2020, the disclosures of which are hereby incorporated by reference in their entireties.

### BACKGROUND OF THE INVENTION

### Field of the Invention

The invention relates to a method for producing a mold element for producing microarrays. The invention further relates to a method for producing a mold element for producing microarrays produced in particular by the method according to the invention.

### Description of Related Art

Microarrays comprise a plurality of microneedles which are typically arranged in or connected to a carrier element such as a patch, a plaster or the like. Microarrays comprise a large number of micro-needles, the length of which is dimensioned such that, when they are pressed into the skin of a patient, they penetrate into the skin only to such an extent that nerves and vessels are not touched by the needle tips, if possible. The needles comprise an active ingredient, for example a medicament. The corresponding active ingredient may be applied on an upper side of the needle or may be provided inside the needles. If the active ingredient is provided inside the needles, the needles or components of the needles are made of a material dissolving in the skin of a patient.

Micro-arrays are produced, for example, using silicone molds having a plurality of recesses. To fill the recesses, a liquid provided with the active ingredient is typically applied on the upper face of the silicone matrix. After the liquid has dried, another liquid is applied, if desired. The carrier element is applied on the lower face of the material placed in the silicone matrix, the microneedles are removed from the silicone matrix and are subsequently packaged.

The production of the silicone matrices is very complex and costly.

### SUMMARY OF THE INVENTION

It is an object of the invention to provide a method for producing a mold element for producing microarrays which is inexpensive and preferably suitable for the production of large quantities. Further, it is an object of the invention to provide a corresponding mold element.

The object is achieved, according to the invention, with a method as described herein and a mold element as described herein, respectively.

In the method for producing a mold element for producing microarrays, a planar base element is first provided. In particular, the base element is a film with a thickness of preferably 0.5-2 mm, particularly preferred 0.5-1.5 mm. Thereafter, recesses are formed in the base element, which is in particular in the form of a film, starting from an upper face of the base element. To form the microneedles, a corresponding material can later be introduced into these recesses. According to the invention, the recesses are opened in a next step on a lower face of the base element opposite the upper face. Thus, the recesses are open both on the upper face and the lower face of the base element. For example, this has the advantage that when filling the recesses with material from above, air can escape from the openings provided on the lower face and the design of the microneedles is thus very precise. In particular, the risk of air bubbles forming in a recess is thereby reduced.

In a particularly preferred of the method according to the invention the recesses are formed by embossing, using an embossing element with protrusions complementary to the recesses. Here, the recesses can be embossed into the base element from the upper face of the base element. Here, the embossing element can be an element designed as a flat element in the form of an embossing board, with the corresponding embossing protrusions being provided on the upper face thereof. It is particularly preferred to use an embossing roller as the embossing element. This has the substantial advantage that it is possible to make the corresponding protrusions in a continuous process. This is particularly advantageous if the base element in the form of a film.

The recesses, in particular all recesses, are preferably opened by heating the base element. Here, it is preferred that the recesses are opened at the lower face by melting. As an alternative, piercing could also be performed, wherein the quality obtained by heating or melting, preferred according to the invention, is considerably higher due to the very small dimensions.

It is particularly preferred that the recess is opened using laser beams. In particular, one laser beam per recess would be directed onto the base element. Here, it is possible to direct the laser beam into the recess from the upper face and to thus form the corresponding opening in the direction of the lower face. As an alternative, the laser beam may also be directed onto the lower face of the base element, so that the recess is opened from the lower face. These two method steps for opening the recess could possibly be combined with each other.

It is particularly preferred that a laser source can be redirected via mirrors in order to form a plurality of recesses in the base element.

By means of the method according to the invention, a large number of mold elements for the production of microarrays can thus be formed very precisely in a continuous process, in particular when a film is used in which the recesses are opened by means of laser beams.

The invention further relates to a mold element for the production of microarrays. It is particularly preferred that the mold element according to the invention is formed using the above described production method and the preferred embodiments of this production method.

The mold element according to the invention has a planar base element. As described above, the base element is preferably in the form of a film which preferably has a thickness in a range from 0.5-2 mm, in particular 0.5-1.5 mm. TPU, PC, PETG are particularly suitable film materials.

Recesses are arranged in the base element, which extend from an upper face of the base element towards a lower face of the base element. Preferably, the recesses are embossed recesses. According to the invention, each recess has an opening. The opening is provided on the lower face of the base element. Each recess thus has two openings opposite to one another.

The cross sections of the recesses preferably tapers from the upper face of the base element towards the lower face. In particular, the recesses are conical or frustoconical in shape. A pyramidal or a truncated pyramidal design of the recesses is particularly preferred. Preferably, the pyramidally designed recess has a square cross section. The recesses are preferably symmetrical in the longitudinal direction, so that the opening provided on their lower face is positioned centrally with respect to a base surface of the recess.

Preferably, each single recess has a cross-sectional area of 0.04 mm²-0.16 mm², in particular of 0.04-0.08 mm², at the upper face. The recesses themselves preferably have a depth of 600 μm to 1500 μm, in particular 600 μm to 1000 μm.

The base element, which is particularly in the form of a film, has a thickness greater than the depth of the recesses. Preferably, the thickness of the film is thicker by 200 μm-500 μm than the depth of the recesses. In particular, the base element has a thickness of 800 μm-2 mm.

The openings on the lower face of the base element are preferably lasered openings, i.e., openings formed using laser beams. Thus, in the area of the openings on the lower face of the base element, the base element has a material structure altered by laser beams. Preferably, in the area of the openings on the lower face of the base element, the base element comprises at least partially melted material.

The openings on the lower face of the base element preferably have a diameter of <40 μm, in particular <10 μm.

Preferably, the recesses have a small spacing or a high density. Specifically, 49-625 recesses per square centimeter are provided in an in particular regular arrangement. Likewise, an arrangement of recesses, e.g., in rows is provided, adjacent rows being arranged with a gap therebetween, respectively.

In a particularly preferred embodiment, the above described preferred size ratios are obtained using the above describes method, so that in a particularly preferred embodiment, the corresponding features, either individually or in combination, can also define the preferred embodiment of the method.

Hereinafter, the invention will be described in more detail by means of a preferred embodiment with reference to the accompanying drawings.

### BRIEF DESCRIPTION OF THE DRAWINGS

In the Figs.:

FIG. 1 is a schematic side view of a base element together with an embossing roller,

FIG. 2 is a schematic side view of the embossed base element together with a laser means, and

FIG. 3 is a schematic enlarged side view of a recess produced by means of the method according to the invention.

### DESCRIPTION OF THE INVENTION

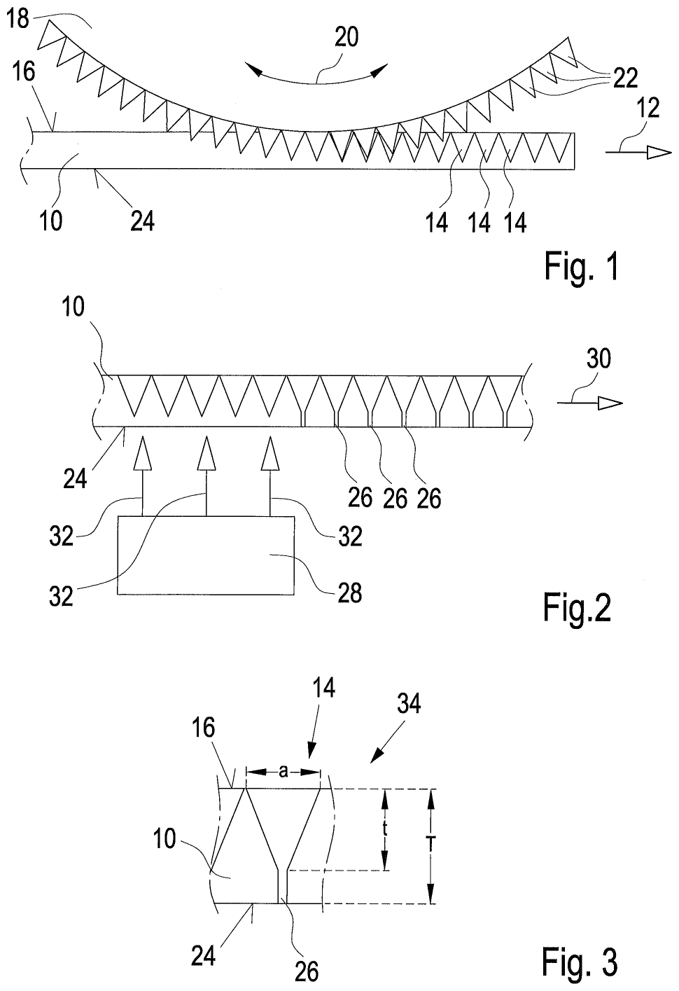

In the embodiment illustrated, a base element 10 in the form of a film is moved from left to right in the direction of an arrow 12 in FIG. 1 to produce a mold element by means of the method according to the invention. To form recesses 14 in an upper face 16 of the base element 10 which, in the embodiment, rotates counterclockwise in the direction of an arrow 20. On an outer face of the embossing roller 18, a plurality of protrusions 22 is provided, the protrusions 22 being arranged such that they are regularly distributed across the entire outer face of the embossing roller 20. The cross section of the protrusions 22 corresponds to the cross section of the recesses 14. The protrusions 22 are in particular formed in a pyramid shape and have a preferably square cross section.

The height of the protrusions 22 and thus the depth of the recesses is slightly smaller than the thickness of the film 10. The film 10 is thus closed on the lower face 24 even after the forming of the recesses 14 by the embossing process.

In a next method step, openings 26 are formed on the lower face 26 of the film 10, the openings being arranged centrally with respect to the recesses 14 (hier lassen sich die beiden deutschen Wörter “zentral bzw. mittig” m. E. nicht vernünftig durch zwei englische Wörter übersetzen). The openings 26 are formed using a laser device 28, while the embossed film 10 is incrementally moved in particular to the right in the direction of an arrow 30 in FIG. 2. The laser device 28 generates in particular a plurality of laser beams 32 which are each directed towards a tip of the recess 14. The openings 26 are formed by means of the laser beams 32.

A mold element 34 produced in particular by means of the method according to the invention comprises a plurality of recesses 14. These are formed such that, starting from the upper face 16 of the base element 10, they taper towards the lower face 24. The recesses 14 each have a pyramidal cross section with an in particular square base surface.

A side length a of the square base surface of the pyramidal recess 14 has dimensions of a=200-400 μm. The depth of the recesses 14 is t 0 600 μm-1500 μm. A diameter of the openings 26 formed by a laser is preferably <40 μm and preferably <10 μm. The total film thickness T is T=800 μm-2 mm.

1. A method for producing a mold element for producing microarrays, the method comprising the steps of:
providing a planar base element,
forming recesses in the base element starting from an upper face of the base element, and
opening the recesses on a lower face opposite the upper face.

2. The method according to claim 1, wherein the base element is in the form of a film having a thickness of 0.5-2 mm, in particular 0.5-1.5 mm.

3. The method according to claim 1, wherein the recesses are formed by embossing.

4. The method according to claim 3, wherein the recesses are made using an embossing roller having a plurality of protrusions complementary to the recesses.

5. The method according to claim 1, wherein the recesses are opened by heating the base element.

6. The method according to claim 1, wherein the recesses are opened by heating or melting the base element.

7. The method according to claim 1, wherein the recesses are opened using laser beams.

8. The method according to claim 7, wherein per recess, one laser beam is directed onto the base element.

9. The method according to claim 8, wherein the laser beams are directed into the recesses from the upper face.

10. The method according to claim 8, wherein the laser beams are directed towards the lower face of the base element.

11. A mold element for producing microarrays, produced in particular by means of the method according to claim 1, comprising
a planar base element,
recesses arranged, in particular embossed, in the base element, which extend from an upper face towards a lower face,
wherein in particular each recess has an opening provided on the lower face of the base element.

12. The mold element according to claim 11, wherein the base element is in the form of a film having a thickness of 0.5-2 mm, in particular 0.5-1.5 mm.

13. The mold element according to claim 11, wherein the base element comprises TPU, PC, PETG.

14. The mold element according to claim 11, wherein the cross sections of the recesses taper from the upper face towards the lower face of the base element.

15. The mold element according to claim 11, wherein the recesses are pyramidal in shape with in particular a square cross section.

16. The mold element according to claim 11, wherein the recesses on the upper face have a cross-sectional area of 0.04 mm²-0.16 mm², in particular of 0.04-0.08 mm².

17. The mold element according to claim 11, wherein the recesses have a depth in a range from 600 μm to 1500 μm, in particular from 600 to 1000 μm.

18. The mold element according to claim 11, wherein the base element has a thickness thicker by 200 μm-500 μm than the depth of the recesses.

19. The mold element according to claim 17, wherein the base element has a thickness of 00 μm to 2 mm.

20. The mold element according to claim 11, wherein the openings are lasered openings.

21. The mold element according to claim 11, wherein the openings have a diameter of <40 μm, in particular <10 μm.
